(19) United States
(12) Patent Application Publication
(10) Pub. No.: US 2018/0325354 A1
(43) Pub. Date: Nov. 15, 2018

(54) SETTING CHANGING APPARATUS FOR ENDOSCOPE

(71) Applicant: OLYMPUS CORPORATION, Tokyo (JP)

(72) Inventor: Takashi SAITO, Tokyo (JP)

(73) Assignee: OLYMPUS CORPORATION, Tokyo (JP)

(21) Appl. No.: 16/035,805

(22) Filed: Jul. 16, 2018

Related U.S. Application Data
(63) Continuation of application No. PCT/JP2017/000995, filed on Jan. 13, 2017.

(30) Foreign Application Priority Data
Jan. 18, 2016 (JP) 2016-006993

(51) Int. Cl. A61B 1/00

(52) U.S. Cl. CPC A61B 1/00009; G16H 30/40; A61B 1/00006

(57) ABSTRACT

A setting changing apparatus for endoscope is provided that, when a surgeon attempts to set setting values to setting values of an old endoscope system including an old endoscope processor, for example, selects system setting and designates an examination region and, thereafter, for example, selects setting of the setting values to latest setting values, reads out an old reference image corresponding to the examination region, sets, as setting values close to the latest setting values, new setting values corresponding to a new reference image such that a deviation amount from the old reference image is equal to or smaller than a threshold, and performs setting of a new endoscope system including a new endoscope processor.

### CROSS REFERENCE TO RELATED APPLICATION

This application is a continuation application of PCT/JP2017/000995 filed on Jan. 13, 2017 and claims benefit of Japanese Application No. 2016-006993 filed in Japan on Jan. 18, 2016, the entire contents of which are incorporated herein by this reference.

### BACKGROUND OF THE INVENTION

### 1. Field of the Invention

The present invention relates to a setting changing apparatus for endoscope that performs setting of an endoscope processor that generates an endoscopic image

### 2. Description of the Related Art

In recent years, endoscopes including image pickup devices have been widely used in a medical field and the like. An image pickup signal obtained by image pickup by an endoscope is converted into an image signal by an endoscope processor and displayed as an endoscopic image on a monitor. A surgeon observes the endoscopic image displayed on the monitor and performs diagnosis of a diseased part and the like.

When the endoscope processor is changed to a new endoscope processor, it is sometimes desired that the new endoscope processor can be used with the same characteristics as characteristics of the old endoscope processor in the past.

For example, International Publication No. 2013/031512 serving as a first conventional example discloses an endoscope system including endoscope processors 13A and 13B, at least a part of settings of which are in common. The endoscope system transmits, on the basis of a predetermined operation, setting content of the endoscope processor 13A determined in higher priority order to the endoscope processor 13B determined in lower priority order and changes, in the endoscope processor 13B, settings common to setting content of the endoscope processor 13B in the received setting content of the endoscope processor 13A to the received setting content of the endoscope processor 13A.

Japanese Patent Application Laid-Open Publication No. 2007-298204 serving as a second conventional example discloses a data transplanting method between new and old units that can transplant data to the new unit without providing storage means for data backup anew even if a remote controller communication function of a remote controller of a water heater is broken.

The data transplanting method between the new and old units includes communication means for performing data communication via an Internet N. In replacing a remote controller of a water heater including a control configuration for periodically transmitting data stored in storage means to a data management server and causing the data management server to accumulate the data, after the replacement of the remote controller, the data transplanting method transmits a lot number of a replaced old remote controller and a lot number of a new remote controller after the replacement to the data management server through a cellular phone and associates the lot numbers and causes, on the basis of the association, the data management server to transmit accumulated data to the new remote controller.

The second conventional example discloses that data of the old remote controller is directly transmitted to the replaced new remote controller to make it possible to use the new remote controller in the same state as the old remote controller and, when the replacement is performed, processing of data such as a format change of the data, deletion of unnecessary data, and addition of new items is performed.

### SUMMARY OF THE INVENTION

A setting changing apparatus for endoscope according to an aspect of the present invention includes: a connecting section to which a first endoscope processor and a second endoscope processor are connected, the first endoscope processor being configured to perform first image processing on an image obtained by observing a first examination region in a subject to generate a first image and perform second image processing, in which a parameter different from a parameter in the first image processing is used, on an image obtained by observing a second examination region, which is an organ different from the first examination region, inside the subject to generate a second image and the second endoscope processor being configured to perform image processing on an inputted image to generate a third image; a recording section configured to record, among information inputted from the first endoscope processor connected to the connecting section, the first image and information concerning the first examination region in association with each other and further record the second image and information concerning the second examination region in association with each other; and an arithmetic processing circuit configured to change a parameter of the image processing in the second endoscope processor connected to the connecting section. When examination of the first examination region is designated in the second endoscope processor, the arithmetic processing circuit compares the first image and the third image recorded in the recording section and changes the parameter of the image processing in the second endoscope processor to be close to a parameter of the first image processing and, when examination of the second examination region is designated in the second endoscope processor, the arithmetic processing circuit compares the second image and the third image recorded in the recording section and changes the parameter of the image processing in the second endoscope processor to be close to a parameter of the second image processing.

### BRIEF DESCRIPTION OF THE DRAWINGS

FIG. 1 is a diagram showing a configuration of a server system for medical use in a first embodiment of the present invention;

FIG. 2 is a diagram showing an internal configuration of one endoscope system in FIG. 1;

FIG. 3 is a diagram showing, in a table format, a setting range of main parameters and a menu range that can be set by a menu in the endoscope system;

FIG. 4 is a diagram showing details of information acquired by a server;

FIG. 5 is a diagram showing, in a table format, models of endoscopes used in the embodiment;

FIG. 6 is a diagram showing, in a table format, an information example acquired by the server, a recommendation example calculated from acquired information by statistical processing, and the like;

FIG. 7 is a diagram showing configurations of an endoscope system connected to the server temporarily earlier and an endoscope system connected to the server temporality later;

FIG. 8 is a flowchart for explaining processing content of a representative operation in the first embodiment;

FIG. 9 is a flowchart for explaining processing for changing parameters such that a difference between an old reference image and a new reference image in FIG. 8 is equal to or smaller than a threshold;

FIG. 10 is a flowchart for explaining processing for statistically calculating recommendation values recommended in a facility size classified according to the number of endoscope processors in use; and

FIG. 11 is a flowchart for explaining processing for further recording information concerning setting values and use information every time when a predetermined period elapses in FIG. 8.

### DETAILED DESCRIPTION OF THE PREFERRED EMBODIMENT

An embodiment of the present invention is explained below with reference to the drawings.

### First Embodiment

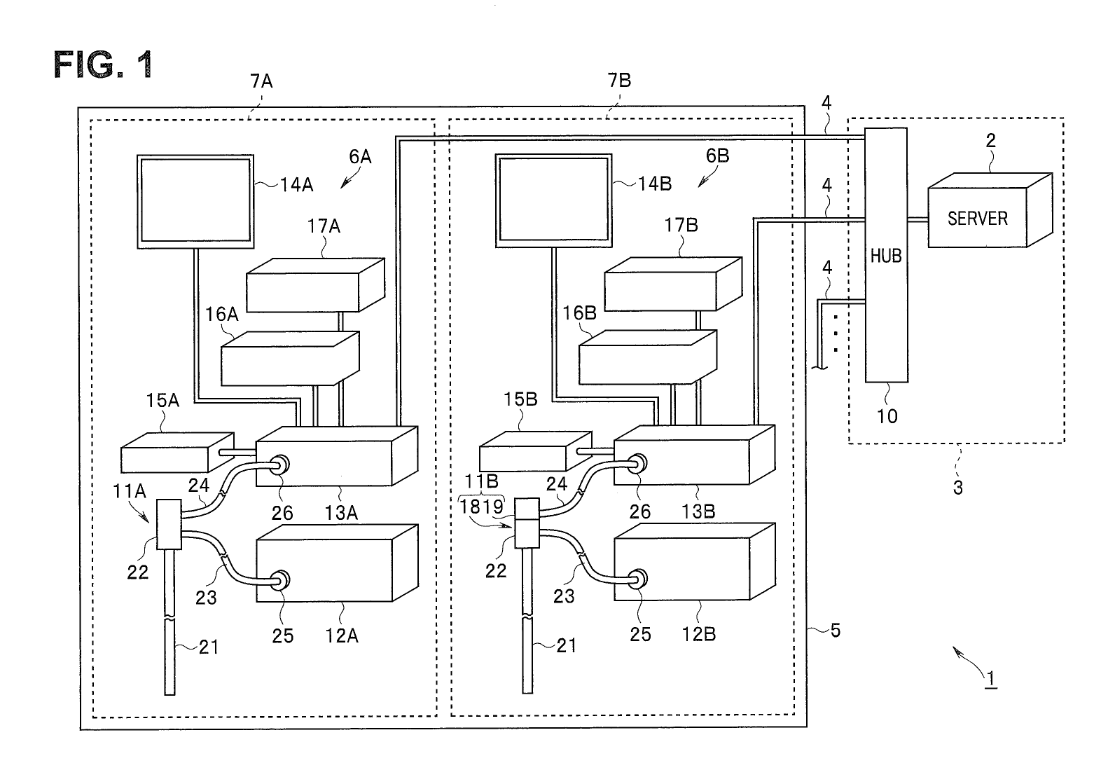

As shown in FIG. 1, a server system for medical use 1 in a first embodiment of the present invention includes a network apparatus 3 including a server 2 disposed in a manufacturer that manufactures an endoscope system and endoscope systems 6A and 6B disposed in a plurality of facilities (in FIG. 1, only one facility is shown) 5 connected to the network apparatus 3 via a communication line 4. In FIG. 1, an example is shown in which the endoscope systems 6A and 6B are respectively disposed in two examination rooms 7A and 7B in one facility 5.

In FIG. 1, the example is shown in which the one facility 5 includes two endoscope systems 6A and 6B. However, it is also possible that a facility like a small hospital having a small size includes only one endoscope system. A facility like a medium-sized hospital having a size larger than a small hospital includes a larger number of endoscope systems than the small hospital. A facility like a large hospital having a size larger than the medium-sized hospital includes a larger number of endoscope systems. Therefore, I in an endoscope system 6I used in the following explanation is not limited to A and B and includes A, B, C, . . . .

The endoscope system 61 includes an endoscope 11I for performing an examination (or an observation), a light source apparatus 12I configured to supply illumination light to the endoscope 11I, an endoscope processor 13I functioning as an image processing apparatus configured to perform signal processing on an image pickup device mounted on the endoscope 11I and generate an image signal (a video signal), a monitor 14I functioning as a display apparatus configured to display an endoscopic image, a keyboard 151 connected to the endoscope processor 13I, and a video tape recorder (abbreviated as VTR) 16I and a printer 17I functioning as peripheral apparatuses. Note that the endoscope system 61 may be configured as an endoscope system including a processor of a light source-integral type in which a light source apparatus is housed inside the endoscope processor 13I.

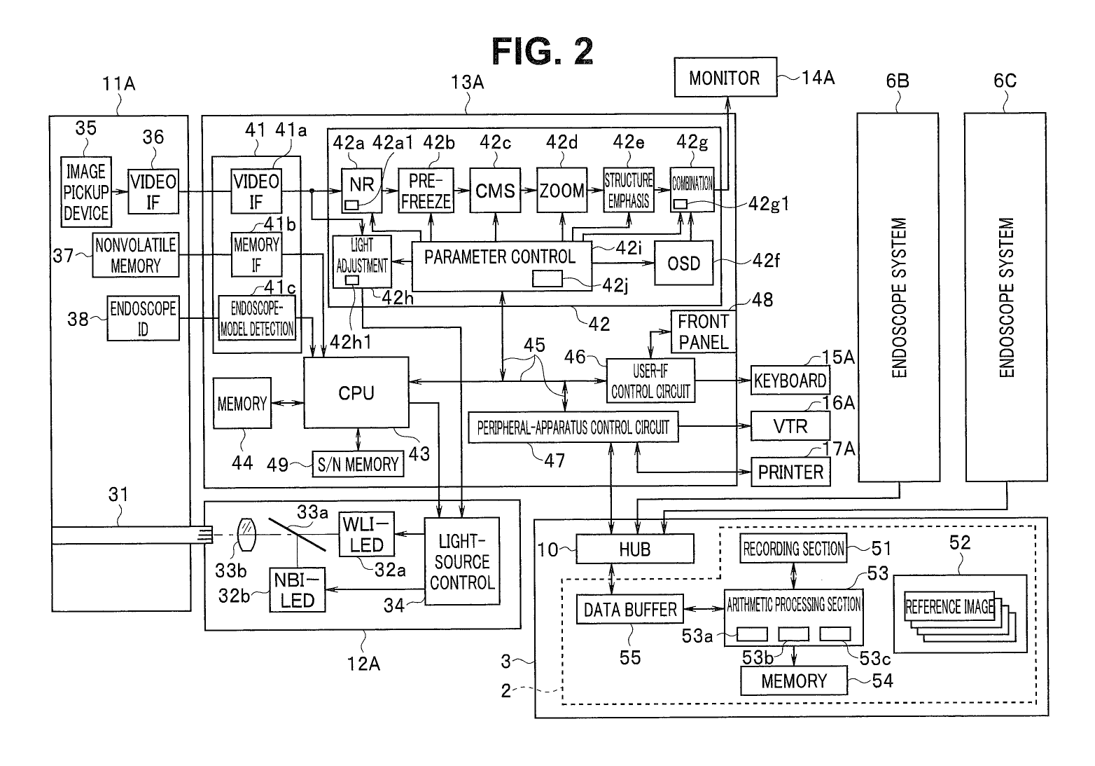

As the endoscope 11I including the image pickup device, there are an endoscope 11A called electronic endoscope incorporating an image pickup device 35 (see FIG. 2) in a distal end portion and a television camera-attached endoscope 11B in which a television camera 19 incorporating an image pickup device is attached to an eyepiece section of an optical endoscope 18 configured by a fiberscope or the like.

The endoscope 11I includes an insertion section 21 inserted into a body of a patient, an operation section 22 provided at a proximal end (a rear end) of the insertion section 21, and a light guide cable 23, a proximal end of which is extended from the operation section 22.

The endoscope 11A includes a signal cable 24, a proximal end of which is extended from the operation section 22. A light source connector 25 and a signal connector 26 at terminal ends of the light guide cable 23 and the signal cable 24 are respectively detachably connected to a light source apparatus 12A and an endoscope processor 13A.

On the other hand, in the case of the endoscope 11B, the proximal end of the signal cable 24 is extended from a television camera 18b. The light source connector 25 and the signal connector 26 at the terminal ends of the light guide cable 23 and the signal cable 24 are respectively detachably connected to a light source apparatus 12B and an endoscope processor 13B.

Note that endoscopes of types, structures themselves of which are different as explained above, and endoscopes of different models (types), outer diameters and the like of the insertion sections 21 of which are set according to an examination region (an observation region or an organ) to be examined (or observed), can be connected to the light source apparatus 12I and the endoscope processor 13I.

The network apparatus 3 includes the server 2 having a function of, for example, recording setting information of the endoscope system 61 and a hub 10 forming a connecting section to which the other end of the communication line 4, one end of which is connected to the endoscope processor 13I, is connected. The endoscope processor 13I is communicably connected to the server 2 (such that transmission and reception of information is possible) via the communication line 4 and the hub 10.

The server 2 acquires various kinds of information of the endoscope processor 13I configuring the endoscope system 61 and saves the acquired various kinds of information.

Therefore, even if the endoscope processor 13I is broken, a new endoscope processor 13I of the same model as the endoscope processor 13I can be connected to the network apparatus 3 and set in the same setting state as the broken endoscope processor 13I.

The server 2 can set setting values of a new endoscope processor connected to the network apparatus 3 to values close to setting values of an old endoscope processor connected to the network apparatus 3 temporally earlier than the endoscope processor.

Even when a model of the endoscope processor 13I used so far is revised, the endoscope processor 13I can be set to a setting state close to a setting state before the revision to make it possible to construct a user-friendly system.

For example, even when the old endoscope processor 13A before the revision is replaced and an endoscope apparatus is formed using a revised new endoscope processor 13A′, in this embodiment, setting of the new endoscope processor 13A′ can be easily performed on the basis of use information of the old endoscope processor 13A before the revision.

FIG. 2 shows a specific configuration of one endoscope system 6A.

As shown in FIG. 2, a light guide 31 for guiding (transmitting) illumination light is inserted through the endoscope 11A. In the light guide 31, the light source connector 25 at the end portion (the terminal end) of the light guide cable 23 is connected to the light source apparatus 12A.

The light source apparatus 12A includes, such that illumination lights corresponding to two observation modes, that is, a wideband observation mode (WLI mode) and a narrowband light observation mode (NBI mode) configuring a special light observation mode can be generated, two light sources, that is, a light emitting diode for wideband observation (abbreviated as WLI-LED) 32a and an LED for narrowband observation (NBI-LED) 32b, a dichroic minor 33a, a condensing lens 33b, and a light-source control circuit 34 configured to control switching of light emission of the two LEDs and to control light emission amounts of the two LEDs.

Note that, in FIG. 2, a configuration example in which the LED is used as the light source apparatus 12A is shown. However, for example, a Xenon lamp may be used and light of the Xenon lamp may be caused to selectively pass through a filter for wideband observation and a filter for narrowband disposed in a circumferential direction of a rotatable disk to generate illumination lights corresponding to the two observation mode. Note that, as the special light observation mode, a fluorescence observation can also be performed by adopting a light source apparatus including a light source that generates excitation light for a fluorescence observation mode.

The WLI-LED 32a generates, for example, white light that covers a visible wavelength band. The white light is mostly transmitted through the dichroic mirror 33a, condensed by the condensing lens 33b, and made incident on the light guide 31 as illumination light for WLI.

The NBI-LED 32b generates, for example, one or two narrowband lights in a blue wavelength band. The narrowband lights are selectively reflected by the dichroic mirror 33a, condensed by the condensing lens 33b, and made incident on the light guide 31 as illumination light for NBI.

The light guide 31 transmits the illumination light made incident from the light source apparatus 12A, emits the illumination light from a distal end face, and illuminates an inside of a body into which (the insertion section 21 of) the endoscope 11A is inserted.

An objective lens (not shown in FIG. 2) that forms an optical image is disposed at a distal end portion of (the insertion section 21 of) the endoscope 11A. An image pickup device 35 that performs photoelectric conversion such as a charge coupled device is disposed in an image forming position of the objective lens.

The endoscope 11A includes a video (or image) interface (in FIG. 2, abbreviated as video IF) 36 to which the image pickup device 35 is connected, the video IF 36 being configured by a buffer circuit or the like, a nonvolatile memory 37 in which information concerning the image pickup device 35 and the like of the endoscope 11A is stored, and an endoscope-ID storing section (in FIG. 2, briefly indicated by endoscope ID) 38 in which endoscope identification information (endoscope ID) including endoscope model information is stored. Note that the endoscope model has the same meaning as an endoscope type or kind.

The signal connector 26 of the endoscope 11A is connected to the endoscope processor 13A, whereby the video interface 36, the nonvolatile memory 37, and the endoscope-ID storing section 38 are connected to an interface circuit 41 in the endoscope processor 13A.

The interface circuit 41 includes a video (or image) interface 41a connected to the video interface 36, a memory interface 41b connected to the nonvolatile memory 37, and an endoscope-model detection circuit 41c connected to the endoscope-ID storing section 38 and configured to detect an endoscope model.

An image pickup signal photoelectrically converted by the image pickup device 35 is converted into a video signal (an image signal) via the video interfaces 36 and 41a and inputted to a video-signal processing circuit (or an image-signal processing circuit) 42 in the endoscope processor 13A. The video interface 41a is configured by, for example, a preamplifier and a correlated double sampling processing circuit (abbreviated as CDS circuit). The video interface 41a converts the image pickup signal inputted via the video interface 36 into an endoscopic video signal (or an endoscopic image signal) and outputs the endoscopic video signal (or the endoscopic image signal) to the video-signal processing circuit 42. Note that the endoscopic video signal (or the endoscopic image signal) is briefly referred to as video signal (or image signal) as well.

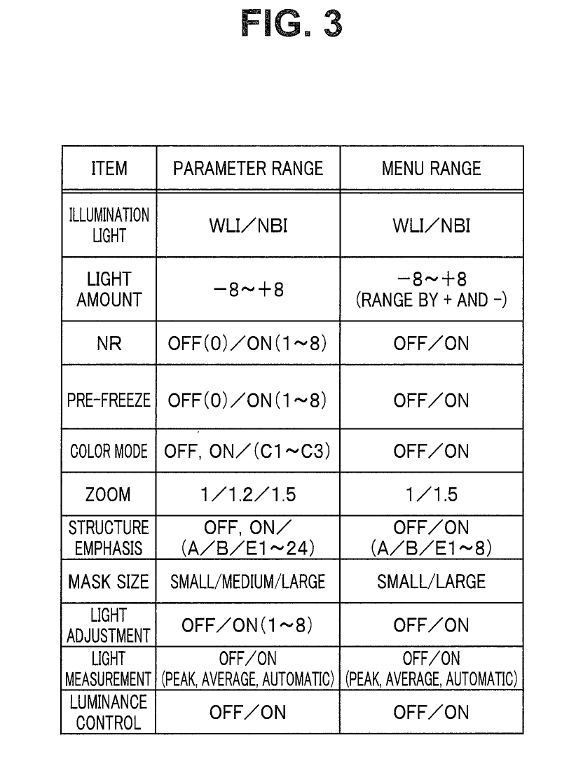

The video-signal processing circuit 42 performs video signal processing (or image signal processing) on the inputted video signal (image signal) with a plurality of circuits having different processing functions and outputs a generated video signal (or image signal) to a monitor 14A functioning as a display device. In this case, the plurality of circuits forming the video-signal processing circuit 42 perform the video signal processing (or the image signal processing) with characteristics corresponding to parameters set to setting values suitable for an examination region (an observation region or an organ) about to be examined or observed. As explained below, FIG. 3 shows an example of parameters of the plurality of circuits forming the video-signal processing circuit 42 (shows parameters other than the parameters of the video-signal processing circuit 42).

Information of the nonvolatile memory 37 is read, via the memory interface 41b, by a central processing unit (abbreviated as CPU) 43 configured to control operation of the endoscope processor 13A.

The endoscope-ID storing section 38 is connected to the CPU 43 via the endoscope-model detection circuit 41c. The CPU 43 acquires information concerning a model of the endoscope 11A connected to the endoscope processor 13A.

Note that the endoscope 11B has structure (not shown in FIG. 2) in which the image pickup device 35, the video interface 36, the nonvolatile memory 37, and the endoscope-ID storing section 38 shown in FIG. 2 are provided in the television camera 19.

In the endoscope processor 13A, a memory 44 connected to the CPU 43 and used to temporarily store various kinds of information and used as a work area, a user-interface control circuit (in FIG. 2, user-IF control circuit) 46 connected to the CPU 43 via a data bus 45, a peripheral-apparatus control circuit 47, and a front panel 48 connected to the user-interface control circuit 46 are provided.

In the endoscope processor 13A, an S/N memory 49 having recorded therein a serial number (in figures such as FIG. 2, abbreviated as S/N) serving as peculiar identification information of the endoscope processor 13A is provided. The S/N memory 49 is connected to the CPU 43. As explained below, the server 2 reads out a serial number of the endoscope processor 13A from the endoscope processor 13A and manages, for each serial number, setting values and use information of an endoscope system connected to the server 2.

A keyboard 15A is connected to the user-interface control circuit 46 together with the front panel 48. The network apparatus 3, a VTR 16A, and a printer 17A are connected to the peripheral-apparatus control circuit 47.

The video-signal processing circuit 42 includes a noise reduction circuit (abbreviated as NR circuit) 42a, a pre-freeze circuit 42b, a color-management-system processing circuit (CMS circuit or color correction circuit) 42c, a zoom circuit 42d, a structure emphasis circuit 42e, an on-screen display circuit (OSD circuit) 42f, a combination circuit 42g, a light-adjusting circuit 42h, and a parameter control circuit 42i.

The NR circuit 42a reduces random noise in a video signal. When a still image frozen by a freeze instruction is displayed, the pre-freeze circuit 42b performs pre-freeze processing before the pre-freeze instruction. The CMS circuit 42c performs color correction processing corresponding to a color mode or a color processing mode.

The zoom circuit 42d performs electronic zoom processing. The structure emphasis circuit 42e performs structure emphasis processing for emphasizing structure such as a contour. The OSD circuit 42f performs processing for displaying a menu screen or the like.

The combination circuit 42g combines the menu screen or the like generated by the OSD circuit 42f with a structure-emphasized video (image). The light-adjusting circuit 42h generates a light adjustment signal. Note that the light-adjusting circuit 42h includes a light measurement circuit 42h1 that detects brightness of an endoscopic image. The parameter control circuit 42i controls parameters of the NR circuit 42a, the pre-freeze circuit 42b, . . . , the light-adjusting circuit 42h, and the light measurement circuit 42h1.

Note that the video-signal processing circuit 42 further includes, for example, in the combination circuit 42g, a mask circuit 42g1 configured to set a mask serving as a size of a display frame in displaying an endoscopic image on the display apparatus. Note that the mask circuit 42g1 may be provided outside the combination circuit 42g. The parameter control circuit 42i also controls parameters for determining a mask size of the mask circuit 42g1.

The video-signal processing circuit 42 includes, for example, in the NR circuit 42a, a luminance control circuit 42a1 configured to perform luminance control (e.g., setting a characteristic of a luminance level of an output signal to be nonlinear with respect to a luminance level of an input signal to control a gradation) such that an endoscopic image can be displayed in a wide gradation. The luminance control circuit 42a1 may be provided on an outside of the NR circuit 42a. The parameter control circuit 42i also controls a parameter concerning whether the luminance control of the luminance control circuit 42a1 is performed (ON/OFF).

FIG. 3 shows a variable range (a parameter range) of parameter in main items in which, for example, functions or characteristics in the endoscope system 6A change and a variable range (a menu range) that can be set by a menu. The menu range is a variable range (a setting range) narrower than the parameter range. Note that FIG. 3 shows one specific example. The variable ranges may include contents different from FIG. 3.

More specifically, there are items such as illumination light and a light amount in the light source apparatus equivalent to the observation mode, NR of the NR circuit 42a in the video-signal processing circuit 42 in the endoscope processor, pre-freeze of the pre-freeze circuit 42b, a color mode of the CMS circuit 42c, zoom of the zoom circuit 42d, negotiating structure emphasis of the structure emphasis circuit 42e, a mask size of the mask circuit 42g1, light adjustment of the light-adjusting circuit 42h, light measurement of the light measurement circuit 42h1, and luminance control of the luminance control circuit 42a1.

As the illumination light (the observation mode), there are (illumination light of) the WLI and (illumination light of) the NBI explained above. Depending on a model of a light source apparatus, the light source apparatus further has a function of generating excitation light corresponding to a fluorescence observation mode for a fluorescence observation. In this case, (the illumination light of) the WLI or (the illumination light of) the NBI can be selected according to a parameter or a menu.

The light amount of the illumination light in the light source apparatus can be adjusted in levels in a wide range of −8 to +8 according to a parameter or a menu. In this case, a level 0 is a reference light amount. The light amount can be set to a light amount of one level from the levels −8 to +8 according to designation of a value of the light amount parameter. Note that, in this embodiment, a larger value of j in a level j (j=−8 to 0 to 8) indicates that a processing function of the respective circuits is larger. Note that, when the light amount is changed according to the menu, the light amount can be variably set by an increase button (+button) and a decrease button (−button).

The noise reduction (NR) by the NR circuit 42a can be changed to levels 1 to 8 as a processing level for reducing random noise at the time when the NR is turned off and at the time when the NR is turned on.

For example, the parameter control circuit 42i turns off the function of the noise reduction of the NR circuit 42a according to a parameter for NR circuit P0 and sets the levels 1 to 8 of the noise reduction according to parameters for NR circuit P1 to P8. The parameter control circuit 42i sets other circuits described below in the same manner. Note that, in the menu, only OFF or ON (a predetermined NR level) of the NR can be set (selected).

Concerning the pre-freeze by the pre-freeze circuit 42b, as shown in FIG. 3, the pre-freeze function can be set to OFF and can be set to any level of the levels 1 to 8. Note that, in the menu, only OFF or ON (a predetermined pre-freeze level) of the pre-freeze can be set (selected).

As the color mode by the CMS circuit 42c, three modes C1, C2, and C3 for performing color correction corresponding to an observation region or a model of an endoscope at the time when the color mode is turned off and at the time when the color mode is turned on are prepared. For example, when an upper digestive tract is observed, the mode C1 for correcting a color of the upper digestive tract to a reddish color is prepared.

When a lower digestive tract is observed, the mode C3 for correcting a color of the lower digestive tract to a greenish color is prepared. The mode C2 is an intermediate mode between C1 and C3. Note that, in the menu, only OFF or ON (e.g., C2) of the color mode can be set (selected).

As a zoom magnification for enlarging an image by electronic zoom processing by the zoom circuit 42d, 1.0 (for turning off the zoom processing), 1.2, and 1.5 can be selectively set. In the menu, only OFF (1) or 1.5 can be set (selected).

Concerning the structure emphasis by the structure emphasis circuit 42e, in observation modes at the time when the structure emphasis is turned off and at the time when the structure emphasis is turned on and A/B/E serving as setting corresponding to a model of an endoscope and the like, a processing level (intensity) of the structure emphasis can be selectively set from different levels 1 to 24 (i.e., A1 to A24/B1 to B24/E1 to E24). In the menu, only OFF and A1 to A8/B1 to B8/E1 to E8, which are rough level setting (compare with the case of the parameter), can be set (selected).

As the mask size by the mask circuit 42g1, one of small, normal, and large can be selectively set as a mask size of an endoscopic image outputted to the display apparatus. In the menu, the mask size can be selected from only small and large.

As the light adjustment by the light-adjusting circuit 42h, automatic light adjustment (Auto) in which a light adjusting function is turned on and OFF (manual) can be selected. In the case of the automatic light adjustment (Auto) in which the light adjusting function is turned on, the light-adjusting circuit 42h generates a light adjustment signal to set brightness of an endoscopic image to an appropriate level. When generating the light adjustment signal, the light-adjusting circuit 42h generates light adjustment signals of levels 1 to 8 and outputs the light adjustment signals to the light-source control circuit 34 of the light source apparatus 12A. In the menu, only OFF or ON (a predetermined light adjustment level) of the light adjustment can be set (selected).

As the light measurement by the light measurement circuit 42h1, brightness can be detected by three light measuring methods of Peak (peak light measurement), Ave (average light measurement), and Auto (automatic light measurement) in the case of OFF in which the light measurement is not performed and in the case of ON in which the light measurement is performed. One light measuring method is designated from the three light measuring methods according to a light measurement parameter. In this case, the light measurement of any one of peak, average, and automatic in the case of OFF and ON of the light measurement can be selected according to the menu.

The peak light measurement is a light measuring method for detecting brightness at a peak value of a video signal. The average light measurement is a light measuring method for detecting brightness at an average value. The automatic light measurement is an intermediate light measuring method obtained by combining the peak light measurement and the average light measurement.

As the luminance control, luminance control ON and OFF can be selected.

A user such as a surgeon can input (select), from the keyboard 15A or the front panel 48 shown in FIG. 2 to the user-interface control circuit 46, setting values or (values of) various parameters as setting information at the time when respective circuits configuring the video-signal processing circuit 42 of the endoscope processor 13A are operated. Note that the information (the parameters) of the items shown in FIG. 3 are included in information acquired by the server 2 explained below. In other words, the server 2 acquires, as information concerning setting values, information concerning parameters of the endoscope system 61 (connected to the server 2).

The user-interface control circuit 46 sends the inputted parameters to the parameter control circuit 42i via the data bus 45. The parameter control circuit 42i performs control for setting parameters of the respective circuits configuring the video-signal processing circuit 42 to be the inputted parameters. The parameters set in the respective circuits configuring the video-signal processing circuit 42 are stored in, for example, a nonvolatile memory 42i1 in the parameter control circuit 42i. Note that a nonvolatile memory 42j may be disposed on an outside of the parameter control circuit 42i.

The video signal outputted from the combination circuit 42g is inputted to the monitor 14A. The monitor 14A displays an image of the video signal as an endoscopic image.

The light-adjusting circuit 42h outputs the generated light adjustment signal to the light-source control circuit 34 of the light source apparatus 12A.

The parameter control circuit 42i is connected to, via the data bus 45, the peripheral-apparatus control circuit 47 connected to the network apparatus 3.

As shown in FIG. 2, the server 2 in the network apparatus 3 includes a recording section 51 configured to record (or save) information concerning setting values and use information of the endoscope system 61 connected via the communication line 4 and an image recording section 52 configured to record (or save), in advance, a plurality of reference images generated by the endoscope processor 13I in the case of at least a representative plurality of setting values (in the endoscope system 61).

Note that, in explanation of operation below, the reference images are briefly explained as being recorded in the image recording section 52 with respect to any parameters. Any reference images having different parameter values may be generated (through interpolation processing or the like) from the representative plurality of reference images (by an arithmetic processing section 53 explained below).

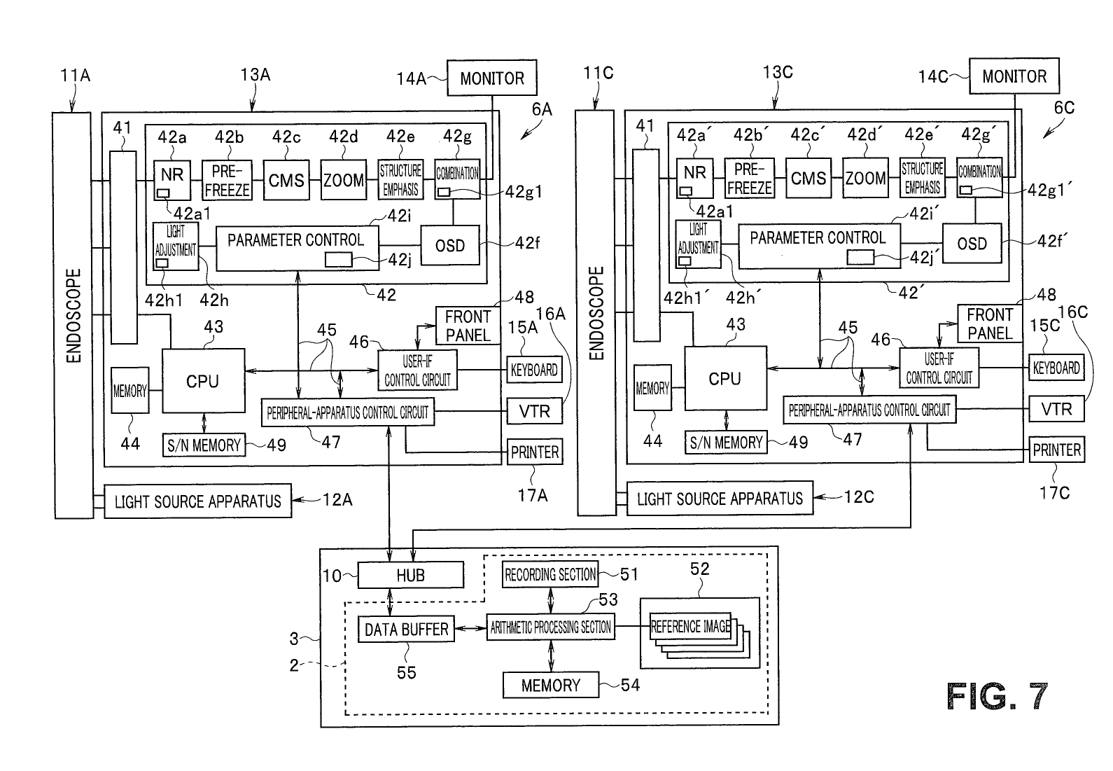

The server 2 includes an arithmetic processing section (or an arithmetic processing circuit) 53 configured to perform arithmetic processing such that deviation between an old reference image serving as a reference image generated by an old endoscope processor in an old endoscope system connected temporally earlier and a new reference image as a reference image generated by a new endoscope processor in a new endoscope system connected temporally later is equal to or smaller than a threshold. FIG. 7 referred to below shows a configuration in which the endoscope system 6A equivalent to the old endoscope system and an endoscope system 6C equivalent to the new endoscope system are connected to the server 2.

Note that the old endoscope system, the old endoscope processor, and the old reference image may be respectively defined as a first endoscope system, a first endoscope processor, and a first reference image. The new endoscope system, the new endoscope processor, and the new reference image may be respectively defined as a second endoscope system, a second endoscope processor, and a second reference image. When setting values of the old endoscope system are represented as old setting values and setting values of the new endoscope system are represented as new setting values, similarly, the old setting values and the new setting values may be respectively defined as first setting values and second setting values.

The arithmetic processing section 53 includes a determination circuit 53a forming a determining section for determining whether a deviation amount between the old reference image and the new reference image is equal to or smaller than the threshold. The arithmetic processing section 53 includes a parameter changing circuit 53b forming a parameter changing section configured to change parameters in the first endoscope system or the second endoscope system.

Note that the parameter changing circuit 53b forming the parameter changing section may be defined to change parameters of the second endoscope system including the second endoscope processor and generate the second setting values such that the deviation amount is equal to or smaller than the threshold.

The determination circuit 53a and the parameter changing circuit 53b are not limited to be provided inside the arithmetic processing section 53 and may be provided outside the arithmetic processing section 53.

When the deviation between the old reference image and the new reference image is larger than the threshold, the arithmetic processing section 53 changes the parameters in the second endoscope system with the parameter changing circuit 53b and performs arithmetic processing such that the deviation is equal to or smaller than the threshold. When the deviation between the old reference image and the new reference image is equal to or smaller than the threshold, the arithmetic processing section 53 performs operation for setting parameters in the new endoscope system in that case as setting values in the new endoscope system.

The arithmetic processing section 53 is formed by a CPU, a DSP (digital signal processor), or the like and has a function of a control section or a control circuit configured to control operation of the server 2. When the endoscope processor 13I configuring the endoscope system 61 is connected to the network apparatus 3, the arithmetic processing section 53 of the server 2 acquires a serial number of the connected endoscope processor 13I via the peripheral apparatus control circuit of the endoscope processor 13I together with other information including setting values and use information and manages the setting values, the use information, and the like of the endoscope processor 13I for each serial number.

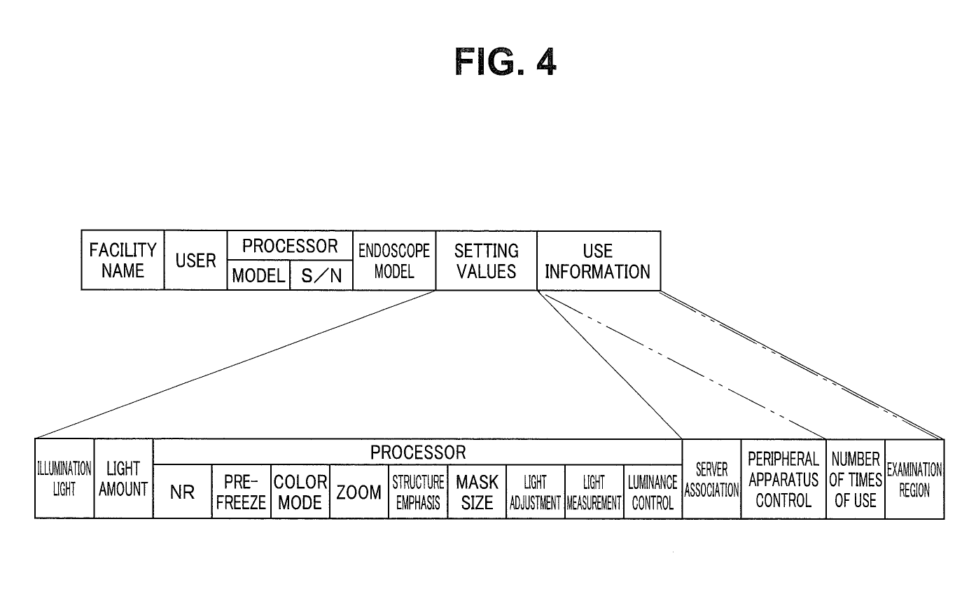

More specifically, as shown in FIG. 4, (the arithmetic processing section 53 of) the server 2 acquires respective kinds of information such as information concerning a facility, a surgeon acting as a user, a model of an endoscope processor, a serial number of the endoscope processor, an endoscope model, and setting values and use information of an endoscope system including the endoscope processor and records the information in the recording section 51.

In FIG. 4, the setting values of the endoscope system include setting values of a light source device and setting values of (mainly the video-signal processing circuit 42 of) the endoscope processor (besides the model of the endoscope).

The server 2 includes a memory 54 used as a work area used when the arithmetic processing section 53 performs arithmetic processing for determining whether deviation between the old reference image and the new reference image is equal to or smaller than the threshold and a data buffer (or a transmitting and receiving section) 55 connected to the hub 10 and configured to perform transmission and reception of information (data) with the endoscope system 61 side connected via the communication line 4.

Note that, in FIG. 2, a state is shown in which three endoscope systems 6A, 6B, and 6C are connected to the hub 10.

As (the information concerning) the setting values, there are mainly setting of illumination light (or illumination light modes corresponding to the observation modes; abbreviated as WLI and NBI) as setting on the light source device side and setting of a light amount of the illumination light.

As (the information concerning) the setting values, setting values of NR, pre-freeze, a color mode (color correction), zoom, structure emphasis, a mask size, light adjustment (mode), light measurement, and luminance control, which are setting on the endoscope processor side, are acquired.

The use information is ON/OFF of server association used in a state in which the endoscope system is connected to the server 2, use information concerning whether a VTR and a printer functioning as peripheral apparatuses are controlled, the number of times of use of the endoscope system, information concerning an examination region in the endoscope system, and the like.

More limited use information in a narrow sense may be limited to information concerning the number of times of use of the endoscope system and the examination region in the endoscope system as indicated by alternate long and two short dashes lines in FIG. 4. Note that the number of times of use represents the number of times of use of the endoscope system in the state in which the endoscope system is connected to the server 2. It is also possible that the endoscope system is used (utilized) for an examination in a state in which the endoscope system is not connected to the server 2. However, the server 2 cannot surely acquire information in that case. Therefore, the use in that case is not included in the number of times of use. However, when the server 2 can acquire information concerning the use of the endoscope system in the state in which the endoscope system is not connected to the server 2, the use may be included in the number of times of use. Note that, as the number of times of use, use for a predetermined time or more may be defined as the number of times of use for one time. In that case, use for time less than the predetermined time is not counted as the number of times of use for one time.

In this embodiment, the server 2 sets at least information concerning the number of times of use of the endoscope system or the examination region in the endoscope system as use information and performs a processing function for setting, on the basis of the use information, setting values of one endoscope processor (e.g., the first endoscope processor) as setting values of another endoscope processor (e.g., the second endoscope processor). More specifically, the arithmetic processing section 53 has a function of a setting processing circuit 53c forming a setting processing section configured to set, on the basis of the use information, setting values of one endoscope processor as setting values of another endoscope processor.

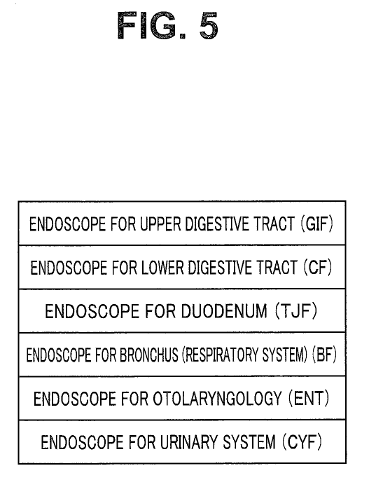

Note that, as explained above, models of endoscopes are prepared according to regions and organs to be examined. Therefore, information concerning the models of the endoscopes is considered to represent a part of the use information. FIG. 5 shows an overview of models of endoscopes usable in this embodiment.

As shown in FIG. 5, as the models of the endoscopes, there are an endoscope for upper digestive tract (GIF), an endoscope for lower digestive tract (CF), an endoscope for duodenum (TJF), an endoscope for bronchus or respiratory system (BF), an endoscope for otolaryngology (ENT), an endoscope for urinary system (CYF), and the like.

In this embodiment, (the arithmetic processing section 53 of) the server 2 calculates, from the acquired information, through statistical processing, recommendation setting values (or recommendation values) recommended in a region to be examined forming the use information and records (saves) the recommendation setting values in the recording section 51.

In this embodiment, the server 2 also has a processing function of setting, on the basis of the use information, setting values of one endoscope processor (e.g., the first endoscope processor) as recommendation values of another endoscope processor (e.g., the second endoscope processor) as explained below.

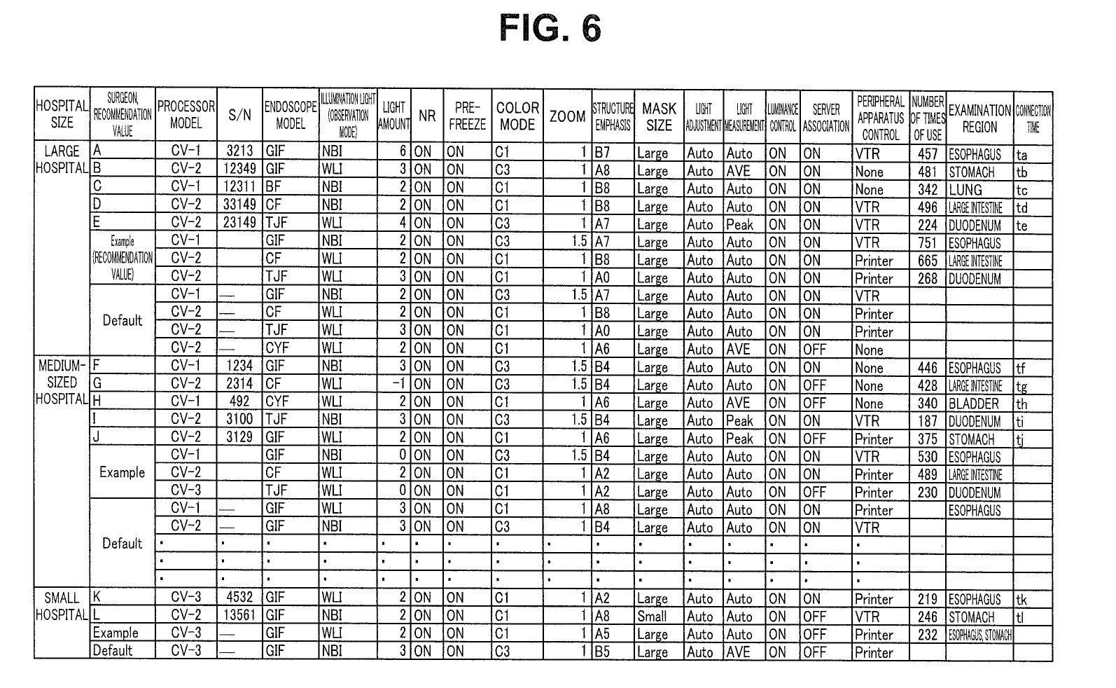

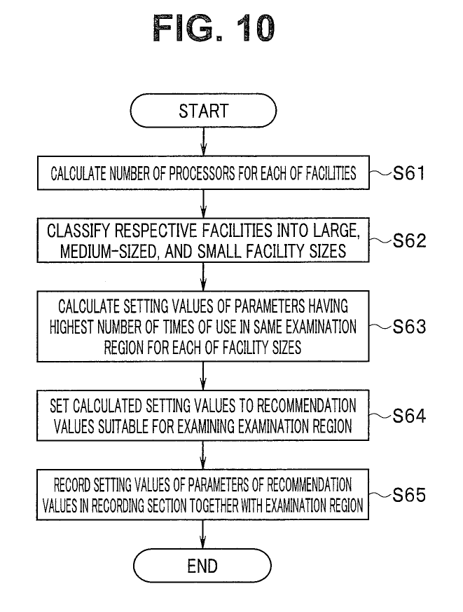

FIG. 6 shows, in a table format, information example acquired by the server 2, recommendation values calculated by statistical processing from the acquired information, and the like. Note that the recommendation values are calculated by, for example, processing shown in FIG. 10 explained below.

As shown in FIG. 6, as items of information acquired by the server 2, the items explained with reference to FIG. 4 are more specifically shown. The server 2 acquires respective kinds of information concerning a hospital name (a hospital size) as information concerning a facility, a surgeon, a model of an endoscope processor, a serial number of the endoscope processor, a model of an endoscope, illumination light (an observation mode), a light amount, NR, . . . , light measurement, luminance control, server association, peripheral apparatus control, the number of times of use, and an examination region, distinguishes the acquired respective kinds of information for each serial number, and records the respective kinds of information in the recording section 51. The server 2 also records information concerning a connection time of the endoscope processor (to the server 2) in the recording section 51. Note that the hospital size is not acquired in the beginning and is treated as the hospital name and is classified by the following statistical processing.

For example, the server 2 acquires information concerning an endoscopic examination of an esophagus set as an examination region performed by a surgeon A in a large hospital using an endoscope processor of a model CV-1 and an endoscope of a model GIF with illumination light set to NBI, a light amount set to 6, NR set to ON, a color mode set to C1, zoom set to 1, structure emphasis set to B7, a mask size set to large, light adjustment and luminance control set to ON, server association set to ON, a VTR associated as peripheral apparatus control, and the number of times of use set to 457.

The server 2 records a connection time ta as well. Note that the connection time ta and the like include information concerning year, month, day, and time (hour and minute) of connection. Therefore, the recording section 51 includes history information acquired when different examination regions and organs are examined (observed) by, for example, an endoscope processor of one model.

For example, information concerning latest setting values concerning an examination region designated by the surgeon or the like can also be specified from the history information (the history information can also be used when setting of a new endoscope processor is performed). More specifically, when the surgeon is about to designate and examine an organ, the surgeon sometimes desires to use, for example, latest setting values examined in the past in the organ. In such a case, by using the history information, it is possible to easily designate an organ (when one endoscope processor is used for examinations of a plurality of different organs), acquire latest setting values in the organ, and set the endoscope processor to the latest setting values.

Note that CV-1, CV-2, and CV-3 serving as the models of the endoscope processors in FIG. 6 briefly indicate high-end, middle, and low-end models.

Concerning the case of other surgeons B to L, the server 2 acquires the same information, distinguishes the acquired respective kinds of information for each of serial numbers, and records the information in the recording section 51.

The arithmetic processing section 53 of the server 2 performs statistical processing on the acquired respective kinds of information, calculates Example (recommendation values) or recommendation setting values in FIG. 6, and records the Example (recommendation values) or the recommendation setting values in the recording section 51 (as recommendation values).

Note that Default in FIG. 6 indicates a setting example of initial values set during factory shipment. The initial values are recorded in the recording section 51.

When performing an endoscopic examination, the surgeon acting as the user can acquire information concerning setting values of a recommended example from the server 2, set the setting values of the endoscope system to the setting values of the recommended example, and perform the endoscopic examination.

When the user desires to reset the setting values of the endoscope system to the initial values during the factory shipment, the user can easily acquire information concerning the initial values from (the recording section 51 of) the server 2 and set the setting values of the endoscope system to the initial values.

FIG. 7 is shows a configuration example in which, for example, the endoscope system 6A shown in FIG. 1 or 2 is connected to the server 2 and a new endoscope system 6C similar to the endoscope system 6A is connected to the server 2 (later than connection time of the endoscope system 6A). In this embodiment, the endoscope system 6C can be set to characteristics close to setting values of the endoscope system 6A.

Note that, in FIG. 7, a configuration of the endoscope system 6A is already explained with reference to FIG. 2. An endoscope 11C, a light source apparatus 12C, a monitor 14C, and the like other than an endoscope processor 13C in the endoscope system 6C have the same configurations as the endoscope 11A, the light source apparatus 12A, the monitor 14A, and the like in the endoscope system 6A.

In the endoscope processor 13C shown in FIG. 7, a video-signal processing circuit 42′ provided in the endoscope processor 13C has a configuration similar to the video-signal processing circuit 42 provided in the endoscope processor 13A. Components such as respective circuits configuring the video-signal processing circuit 42′ are indicated by adding an apostrophe (') to the NR circuit 42a, the pre-freeze circuit 42b, . . . , the parameter control circuit 42i, the nonvolatile memory 42j, and the like in the video-signal processing circuit 42.

A variable range of parameters in, for example, the structure emphasis circuit 42e in the endoscope processor 13A configuring the endoscope system 6A is A/B/E1 to 24 as shown in FIG. 3. On the other hand, a variable range of parameters in, for example, the structure emphasis circuit 42e′ in the endoscope processor 13C is different. The variable range of the parameters in, for example, the structure emphasis circuit 42e′ may be, for example, a rougher variable range (e.g., A/B/E1 to 16).

Even when the variable range of the parameters is different in this way, setting values of the endoscope system 6C can be set to a state close to the setting values of the endoscope system 6A. By setting both the setting values of both the endoscope systems 6A and 6C to the close state, setting values of the endoscope processor 13C in the endoscope system 6C can be set to a state close to the setting values of the endoscope processor 13A in the endoscope system 6A.

Note that, in this embodiment, as explained below, when the setting values of the endoscope system 6C are set to the state close to the setting values of the endoscope system 6A, setting considering an examination region forming use information can be performed.

When the surgeon attempts to examine, for example, an esophagus using the endoscope system 6C, it is more desired to set the setting values to, for example, setting values close to latest setting values at the time when the same esophagus is examined in the endoscope system 6A than setting the setting values to a state (setting values) close to setting values at the time when a stomach different from the esophagus is examined.

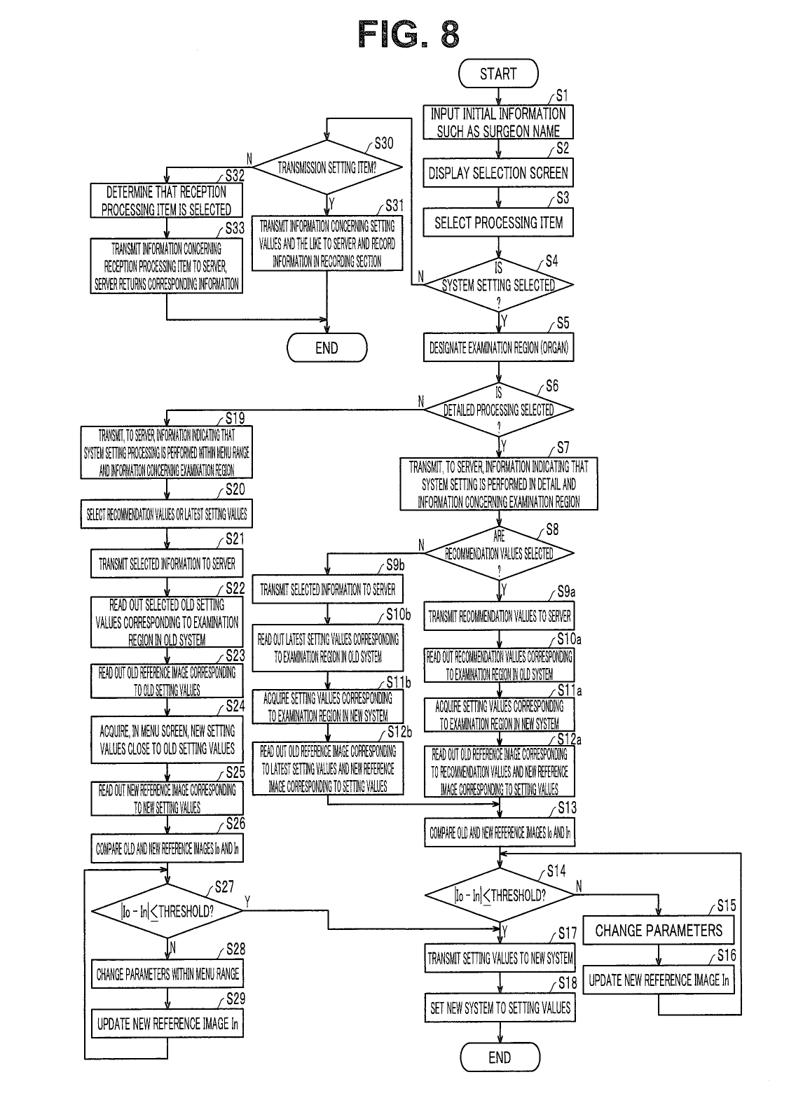

For example, when present setting values of the endoscope system 6A are setting values at the time when the stomach is examined, it is desired to set the setting values to, for example, a state (or setting values) close to latest setting values set when the same examination region (organ) to be examined is examined using the endoscope system 6C before the present (in the past). In this embodiment, setting processing conforming to such a request is performed (explained below with reference to FIG. 8).

In this embodiment, when an examination region (organ) to be examined is examined using the endoscope system 6C, selection for setting the setting values to setting values close to the recommendation values in the endoscope system 6A can also be performed.

The server system for medical use 1 in this embodiment includes the hub 10 forming a connecting section configured such that a first endoscope processor (e.g., 13A) and a second endoscope processor (e.g., 13C) are connected, the recording section 51 configured to record first setting values set in processing in a first endoscopic image signal inputted from the first endoscope processor connected to the connecting section and use information including a use history of the first endoscope processor, and the arithmetic processing section 53 forming a setting processing section configured to generate, on the basis of the first setting values and the use information recorded in the recording section 51, second setting values set in processing in a second endoscopic image signal of the second endoscope processor connected to the connecting section after the connection of the first endoscope processor.

A representative operation of the server system for medical use 1 in this embodiment is explained. For example, a setting operation for setting the setting values of the endoscope system 6C in FIG. 7 to new setting values (second setting values) to be set and setting the new setting values to a state close to the old setting values (the first setting values) of the endoscope system 6A is explained.

In the following explanation, the endoscope system 6A is referred to as old endoscope system (first endoscope system) as well and the endoscope system 6C is referred to as new endoscope system as well.

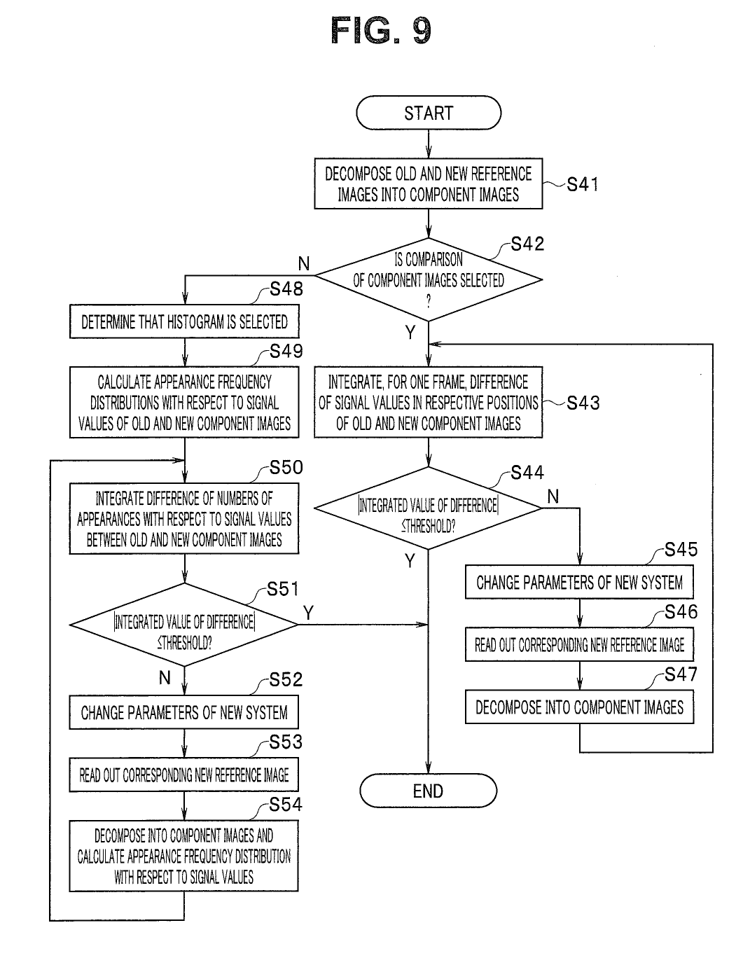

FIG. 8 shows representative processing including a system setting operation in the server system for medical use 1. Note that, in FIG. 8 (in FIGS. 9 and 10 as well), the endoscope system and the endoscope processor are respectively abbreviated as system and processor.

As shown in FIG. 2 or FIG. 7, when the endoscope system 6C is connected to the server 2 and a power supply of the endoscope system 6C is turned on, the endoscope system 6C comes into an operation state. In first step S1, the surgeon acting as the user who uses the endoscope system 6C inputs initial information such as a surgeon name and sex and age of a patient to be examined. Note that, in step S1, the surgeon may input information concerning an examination region (an organ to be examined).

In next step S2, the CPU 43 of the endoscope system 6C displays a selection screen for processing items (work items) such that the surgeon can smoothly perform an examination.

In step S3, the surgeon selects, referring to the selection screen, with a keyboard 15C or the like, processing items about to be performed.

As the processing items, a system setting processing item for setting the new endoscope system 6C to a state close to the setting values of the old endoscope system 6A, a transmission processing item for transmitting setting value information and the like to record setting values and the like of the endoscope system in the recording section 51 of the server 2 as recording information, and a reception processing item for reading out and receiving the recorded recording information. Besides, in the case of the new endoscope processor 13C of the same model as the old endoscope processor 13A, a copy setting processing item for setting the new endoscope processor 13C to the same setting values as the setting values of the old endoscope processor 13A may be provided.

In the case of the copy setting processing item, the new endoscope processor 13C only has to read out the setting values of the old endoscope processor 13A from the recording section 51 and set the setting values of the new endoscope processor 13C to the read-out setting values (not shown in FIG. 8).

Note that, in this embodiment, even when the old endoscope processor 13A breaks down, because information of the old endoscope processor 13A and the old endoscope system 6A is recorded in the recording section 51, it is also possible to set the setting value of the new endoscope processor 13C of the same model as the old endoscope processor 13A to the same setting values as the setting values of the old endoscope processor 13A.

In step S4, the CPU 43 of the endoscope system 6C determines whether system setting is selected as the processing item. When the CPU 43 determines that the system setting is selected as the processing item, in next step S5, the surgeon designates an examination region (an observation part or organ) actually about to be examined in the endoscope system 6C.

In next step S6, the CPU 43 of the endoscope system 6C determines whether detailed processing for performing setting in detail within a settable parameter range in the endoscope system 6C is selected.

When the surgeon desires to perform the setting in the detailed processing, the surgeon selects the detailed processing. When the surgeon desires to perform the setting within a menu range in which simple processing is performed, the user selects the menu range.

When the detailed processing is selected by the surgeon, in step S7, the CPU 43 of the endoscope system 6C transmits information indicating that the system setting is performed in the detailed processing and information concerning the examination region (organ) to the arithmetic processing section 53 of the server 2.

In this embodiment, the surgeon can select processing for setting the new endoscope system 6C to setting values close to one of recommended recommendation values (serving as setting values) in the old endoscope system 6A and latest setting values (corresponding to a designated examination region) in the old endoscope system 6A. Besides, the surgeon may be able to select other setting values (e.g., setting values of a specific surgeon) different from the latest setting values or input information concerning conditions and select setting values satisfying the conditions.

In next step S8, the CPU 43 of the endoscope system 6C determines whether (setting to) the recommendation values is selected by the surgeon. When the recommendation values are selected, the CPU 43 transmits information indicating that the recommendation values are selected to the arithmetic processing section 53 of the server 2 as shown in step S9a. Note that the processing in step S8 may be performed before step S7. In step S7 following step S8, the processing of step S9a may be performed together.

In step S10a following step S9a, the arithmetic processing section 53 reads out, on the basis of the information concerning the examination region and the information concerning the recommendation values, recommendation values (system recommendation values) in the old endoscope system 6A from the recording section 51 when the examination region is examined and in facility (in this embodiment, facility 5) sizes of both the endoscope systems 6A and 6C. In FIG. 6, an item described as Example (recommendation values) corresponds to the recommendation values. For example, when the facility of the endoscope systems 6A and 6C is a large hospital, the model of the endoscope processor is CV-2, and a large intestine is designated as the examination region, setting values of recommendation values corresponding to such conditions are read out from the recording section 51.

In next step S11a, the arithmetic processing section 53 acquires, via the CPU 43 or the peripheral-apparatus control circuit 47 of the new endoscope system 6C, for example, present setting values (system setting values) of the new endoscope system 6C at the time when the examination region is examined.

Note that, when the server 2 side acquires setting values at the time when the new endoscope system 6C is connected to the server 2 and records the setting values in the recording section 51 (together with an acquisition time), the present setting values (system setting values) can also be acquired from the recording section 51. The setting values acquired in step S11a are highly likely to be finally updated by recommendation values (when deviation from the recommendation values is large). Therefore, setting values other than the present setting values may be acquired.

In step S11a, (parameters of) the setting values close to (parameters of) the recommendation values may be set as the setting values of the new endoscope system 6C to reduce the number of times of processing for changing the parameters.

In the explanation with reference to FIG. 7, a changeable parameter range in the new endoscope processor 13C and a changeable parameter range in the old endoscope processor 13A are explained as partially different. However, it is also possible that the parameter ranges are the same.

For example, when the model of the new endoscope processor 13C is CV-2, the model of the old endoscope processor 13A in the recommendation values is the same CV-2, and the parameter ranges of both of the new endoscope processor 13C and the old endoscope processor 13A coincide, if the parameters of the new endoscope processor 13C are matched to parameter values of the recommendation values, the setting values of the new endoscope system 6C can be matched to the recommendation values by reducing the number of times of change of the parameters (to zero).

Therefore, the setting values of the new endoscope processor 13C can also be easily set to the recommendation values of the old endoscope processor 13A of the same model as, for example, a broken new endoscope processor 13C (note that this is not limited to the case of the broken endoscope processor). The setting values of the new endoscope processor 13C can also be set to setting values close to the recommendation values when a parameter range and a model are different as explained below. The setting values of the new endoscope processor 13C can also be set to setting values close to the recommendation values when, even in the same model, characteristics and a parameter range of an old model are changed according to a change of a circuit, an integrated circuit, or the like to be manufactured. Note that, when the setting values of the new endoscope processor 13C are set to (latest) setting values explained below (rather than selecting the recommendation values), the setting values can be set to setting values (equal to or smaller than a threshold) close to the (latest) setting values in the same manner

In next step S12a, the arithmetic processing section 53 reads out, from the image recording section 52, a reference image (referred to as old reference image) at the time when the examination region is examined with the recommendation values by the old endoscope system 6A and reads out, from the image recording section 52, a reference image (referred to as new reference image) at the time when the same examination region is examined by the new endoscope system.

As explained above, when the old reference image and the new reference image are not recorded in the image recording section 52, the arithmetic processing section 53 may perform processing for generating an old reference image of the recommendation values and a new reference image of the setting values at the time values of a part of parameters are changed from a recorded old reference image and a recorded new reference image.

In next step S13, the arithmetic processing section 53 compares the old reference image and the new reference image.

In next step S14, (the determination circuit 53a of) the arithmetic processing section 53 determines whether a condition that an absolute value of a difference between an old reference image Io and a new reference image In is equal to smaller than a threshold is satisfied.

When the condition that the absolute value of the difference between the old reference image Io and the new reference image In is equal to or smaller than the threshold is not satisfied, in step S15, the arithmetic processing section 53 changes parameter values in the setting values of the new endoscope system 6C. Further, in step S16, the arithmetic processing section 53 reads out the new reference image In corresponding to setting values (system setting values) of the changed parameter values from the recording section 51. The arithmetic processing section 53 performs the processing in step S14 using the new reference image In.

As explained with reference to FIG. 7, when the parameter range of the structure emphasis is different, even if the parameter values of the structure emphasis are matched, a level difference between adjacent parameter values is different depending on a variable number or the like of parameters. Therefore, the condition that the absolute value of the difference between the old reference image Io and the new reference image In is not sometimes satisfied. When the condition is not satisfied in this way, as explained above, the parameter values are changed, the corresponding new reference image In is read out from the recording section 51, and the processing in step S14 is performed.

By repeating the processing in steps S14 to S16, the condition that the absolute value of the difference between the old reference image To and the new reference image In is equal to or smaller than the threshold (e.g., Th1) can be set to be satisfied. Note that, even when this condition is satisfied, when the models or the parameter variable ranges of the old endoscope processor 13A and the new endoscope processor 13C are different, the same processing may be further performed by changing the parameters to calculate the new reference image In that satisfies a condition that the absolute value of the difference between the old reference image Io and the new reference image In is minimized.

When determining in step S14 that the condition is satisfied, in step S17, the arithmetic processing section 53 transmits setting values (system setting values) in the case of the new reference image In satisfying the condition to the CPU 43 of the endoscope system 6C.

In step S18, the CPU 43 of the endoscope system 6C sets the endoscope system 6C to the transmitted setting values (system setting values) and ends the processing shown in FIG. 8.

When the recommendation values are not selected in step S8 and, for example, latest setting values (corresponding to the examination region) are selected, in step S9b, the CPU 43 of the endoscope system 6C transmits information concerning the selected latest setting values to the arithmetic processing section 53 of the server 2.

In step S10b, the arithmetic processing section 53 reads out, on the basis of the information concerning the examination region (already transmitted), from the recording section 51, latest setting values (more specifically, system setting values) set in the old endoscope system 6A at the time when the examination region is examined.

In step S11b, the arithmetic processing section 53 acquires, via the CPU 43 or the peripheral-apparatus control circuit 47 of the new endoscope system 6C, for example, present setting values (system setting values) of the new endoscope system 6C at the time when the examination region is examined.

Note that, similarly to the case explained in step S11a, the arithmetic processing section 53 may set (parameters of) the setting values close to (parameters of) the latest setting values to the setting values of the endoscope system 6C and reduce the number of times of processing for changing the parameters.

In next step S12b, the arithmetic processing section 53 reads out, from the image recording section 52, a reference image (referred to as old reference image) at the time when the examination region is examined with the latest setting values by the old endoscope system 6A and reads out, from the image recording section 52, a reference image (referred to as new reference image) corresponding to setting values in step S11b at the time when the same examination region is examined by the new endoscope system.

After the processing in step S12b, the arithmetic processing section 53 proceeds to processing in step S13. The processing explained above is performed. When the condition in step S14 is not satisfied, the processing in steps S15 and S16 is performed. When the condition in step S14 is satisfied, the processing in steps S17 and S18 is performed. The processing shown in FIG. 8 is ended.

When the simple processing performed within the menu range is selected in step S6 rather than the detailed processing, in step S19, the CPU 43 of the endoscope system 6C transmits information indicating that the processing is performed within the menu range and information concerning the examination region (organ) to the arithmetic processing section 53 of the server 2.

In next step S20, when the system setting processing is performed, the CPU 43 of the endoscope system 6C determines whether the recommendation values are selected by the surgeon or the latest setting values are selected by the surgeon.

In next step S21, the CPU 43 of the endoscope system 6C transmits, to the arithmetic processing section 53 of the server 2, selected one kind of information in the recommendation values (the recommendation setting values) or the latest setting values selected in immediately preceding step S20.

In next step S22, the arithmetic processing section 53 reads out, from the recording section 51, information concerning setting values (referred to as old setting values) of the old endoscope system 6A corresponding to the examination region and corresponding to the selected information (according to transmission information in step S19 and step S21).

In next step S23, the arithmetic processing section 53 reads out the old reference image corresponding to the old setting values from the image recording section 52. In next step S24, the arithmetic processing section 53 acquires, within the menu range (in the endoscope system 6C), for example, setting values (referred to as new setting values) close to the old setting values in the new endoscope system 6C.

The processing explained above (e.g., steps S9a to S12a and steps S13 to S16) is performed in the parameter range shown in FIG. 3. However, in this case, substantially the same processing is performed in the menu range. However, in the menu range, a changing range of the parameters in the respective items is small (compared with the case of the parameter range). There are many items such as only OFF/ON. Therefore, for example, when a parameter of a certain item in the old setting values is, for example, ON, the parameter of the same item is set to ON closer than OFF as new setting values.

Note that, when it is difficult to perform setting of new setting values close to old setting values in a certain item, the new setting values may be set to any setting values settable within the menu range or present setting values. Even in such a case, the new setting value can be appropriately set according to processing explained below.

In next step S25, the arithmetic processing section 53 reads out a new reference image corresponding to the new setting values from the image recording section 52.

In next step S26, the arithmetic processing section 53 compares the old reference image Io and the new reference image In.

In next step S27, the arithmetic processing section 53 determines whether a condition that the absolute value of the difference between the old reference image Io and the new reference image In is equal to or smaller than a threshold Th2 is satisfied.

When the condition is not satisfied, in next step S28, the arithmetic processing section 53 changes the parameters within the menu range. Further, in step S29, the arithmetic processing section 53 updates the new reference image In according to the change of the parameters. The arithmetic processing section 53 returns to the processing in step S27.

In this way, when the condition in step S27 is satisfied, the processing shifts to the processing in step S17. In this case, the arithmetic processing section 53 transmits, to the new endoscope system 6C, setting values within the menu range corresponding to the new reference image In satisfying the condition in step S27. The new endoscope system 6C is set to the transmitted setting values and ends the processing shown in FIG. 8.

Note that the old and new reference images are used in the processing in steps S19 to S29. However, when both parameters are associated with the characteristics such that mutual change amounts of characteristics at the time when the parameters are changed can be estimated between the setting values in the menu range of the old endoscope system 6A and the setting value in the menu range of the new endoscope system 6C, the arithmetic processing section 53 may determine from the change of the parameters of the new endoscope system 6C (without using the reference images) whether a deviation amount between both the setting values is equal to or smaller than a predetermined threshold.

In step S4, the surgeon can select the transmission processing item or the reception processing item rather than the system setting processing item.

When the transmission processing item is selected, as shown in step S31, the CPU 43 of the endoscope system 61 transmits information concerning the setting values and the like of the endoscope system 61 to the arithmetic processing section 53 of the server 2. The arithmetic processing section 53 records the transmitted information concerning the setting values and the like in the recording section 51.

When a processing item, which is not the transmission processing item, is selected in step S30, as shown in step S32, the CPU 43 of the endoscope system 61 determines that the reception processing item is selected. As shown in next step S33, the CPU 43 of the endoscope system 61 transmits information of the reception processing item to the arithmetic processing section 53 of the server 2. The arithmetic processing section 53 transmits the information of the reception processing item such that the CPU 43 of the endoscope system 61 can receive the information of the reception processing item.

Note that the information of the transmission processing item and the reception processing item may be the information shown in FIG. 6 or may be information described below, that is, information concerning setting values of, for example, the endoscope processor 13I connected to the server 2, the endoscope 11I used together with the endoscope processor 13I, models of the endoscope processor 13I and the endoscope 11I, a serial number of the endoscope processor 13I, a name of a facility in which the endoscope processor 13I is set, a name of a user (a name of a surgeon) who uses the endoscope processor 13I, the number of times of use included in use information of the endoscope processor 13I, information concerning an examination region (organ), and the like. A part of these kinds of information can be selected.

FIG. 9 shows a more specific processing example of steps S13 to S16 in FIG. 8. When processing for comparing an old reference image and a new reference image is started, in first step S41, the arithmetic processing section 53 decomposes the old reference image Io and the new reference image In respectively into old reference component images Io1 and Io2 and new reference component images In1 and In2 as component images.

Note that the old reference component images Io1 and Io2 and the new reference component images In1 and In2 indicate one example. The component images could be old reference component images Io1, Io2, and Io3 and new reference component images In1, In2, and In3, each of which are three component images, or an old reference component image Io1 and a new reference component image In1, each of which is only one component image.

For example, in the case of the WLI, the component images are three component images of red, green, and blue. In the NBI, the component images are usually two or one component image (are sometimes three component images).

In next step S42, the arithmetic processing section 53 determines whether direct comparison of signal values (pixel values) of the component images is selected by the surgeon to be performed.

In this embodiment, the comparison can be selected to be performed when signal values in two-dimensional positions of the old reference component images Io1 and Io2 and the new reference component images In1 and In2 are set as respective feature values (old feature values and new feature values) and when the signal values are set as feature values of an entire image, the feature values of which are histograms of images for one frame. Note that only one of the feature values may be prepared to perform the comparison.

When the direct comparison of the signal values (pixel values) of the component images is selected to be performed, in step S43, the arithmetic processing section 53 respectively calculates integrated values of differences in the same two-dimensional position between the old reference component Images Io1 and Io2 and the new reference component images In1 and In2 for one frame.

In next step S44, the arithmetic processing section 53 determines whether absolute values of the integrated values of the respective differences are respectively equal to or smaller than a threshold.

In the case of a determination result that the absolute values of the integrated values of the respective differences are respectively not equal to or smaller than the threshold, in step S45, the arithmetic processing section 53 changes the parameters of the new endoscope system 6C. In step S46, the arithmetic processing section 53 reads out the new reference image In corresponding to the changed parameters from the recording section 51.

In step S47, the arithmetic processing section 53 decomposes the new reference image In into component images and thereafter returns to the processing in step S43 and repeats the processing explained above. In the case of a determination result that the absolute values of the integrated values of the respective differences are respectively equal to or smaller than the threshold in step S44, the arithmetic processing section 53 ends the processing shown in FIG. 9 and shifts to the processing in step S17 shown in FIG. 8.

When the direct comparison of the signal values is not selected in the processing in step S42, in step S48, the arithmetic processing section 53 determines that the comparison of the histograms is selected.

In step S49, the arithmetic processing section 53 calculates histograms of appearance frequency (number of appearances) distributions with respect to the signal values of the respective component images in the old endoscope system 6A and the new endoscope system 6C.

In step S50, the arithmetic processing section 53 integrates differences of appearance frequencies (numbers of appearances) in a plurality of signal values in histograms of appearance frequency (number of appearances) distributions of respective old and new component images in the old and new endoscope systems 6A and 6C.

In step S51, the arithmetic processing section 53 determines whether absolute values of the differences of the appearance frequencies (the numbers of appearances) in the plurality of signal values between the old and new component images are respectively equal to or smaller than the threshold.

In the case of a determination result that the absolute values of the integrated values of the respective differences are respectively not equal to or smaller than the threshold, in step S52, the arithmetic processing section 53 changes the parameters of the new endoscope system 6C. In step S53, the arithmetic processing section 53 reads out a new reference image corresponding to the changed parameters from the recording section 51.

In step S54, the arithmetic processing section 53 decomposes the new reference image into component images and further calculates a distribution of appearance frequencies with respect to signal values in the respective component images and thereafter returns to the processing in step S50 and repeats the processing explained above. In the case of a determination result that the absolute values of the integrated values of the differences of the respective appearance frequencies are respectively equal to or smaller than the threshold in step S51, the arithmetic processing section 53 ends the processing shown in FIG. 9 and shifts to the processing in step S17 shown in FIG. 8.

FIG. 10 shows a processing example in which the arithmetic processing section 53 of the server 2 generates recommendation values.

In step S61, the arithmetic processing section 53 calculates, referring to the information recorded in the recording section 51, the number of endoscope processors used in respective facilities (hospitals) from serial numbers of the endoscope processors.

In step S62, the arithmetic processing section 53 classifies the respective facilities into three facility sizes of a large facility, a medium-sized facility, and a small facility using thresholds Th1 and Thm (Th1>Thm) of the number of endoscope processors corresponding to sizes of the facilities. The arithmetic processing section 53 classifies, in FIG. 10, the facilities into the sizes of the facilities and calculates statistical recommendation values for each of the classified sizes reflecting a tendency that setting values in the case of an examination are different depending on the sizes of the facilities. Note that the arithmetic processing section 53 may calculate recommendation values for each of the respective facilities.

In step S63, the arithmetic processing section 53 calculates, for each of the same facility sizes, setting values of parameters having the highest number of times of use in the same examination region. In this case, the arithmetic processing section 53 may calculate setting values of parameters having the highest number of times of use in a designated period. For example, when a period from a certain period in the past to the present is designated, the arithmetic processing section 53 calculates, in the designated period, for each of the same facility sizes, setting values of parameters having the highest number of times of use in the same examination region.

In step S64, the arithmetic processing section 53 sets the setting values of the parameters having the highest number of times of use calculated in preceding step S63 as setting values of parameters of recommendation values recommended in the examination region.

In step S65, the arithmetic processing section 53 records the setting values of the parameters of the recommendation values in the recording section 51 together with the examination region and enables information concerning the setting values to be referred to (read out). The arithmetic processing section 53 ends the processing shown in FIG. 10.

Note that, when the endoscope processor 13I is connected to the server 2 first in processing shown in FIG. 8, the arithmetic processing section 53 may record setting values and use information of the endoscope system 61 including the endoscope processor 13I in the recording section 51 together with time when the endoscope processor 13I is connected (information concerning the connection).

After the processing in step S18, after the processing in step S29, after the processing in step S31, and after the processing in step S33 in FIG. 8, for example, the arithmetic processing section 53 may add setting values and use information recorded in the recording section 51 every time a predetermined period elapses from time (a period) when the setting values and the use information (of the endoscope system 61) are recorded in the recording section 51.

That is, the arithmetic processing section 53 may read out time information concerning the latest time when the setting values and the use information are recorded in the recording section 51 in the endoscope system 61 connected to the server 2, determine whether a predetermined period (a predetermined time) set by the surgeon or the like has elapsed from the time of the time information, and, when the predetermined period has elapsed, record the setting values and the use information of the endoscope system 61 including the endoscope processor 13I in the recording section 51 together with time information (in recording the setting values and the use information).

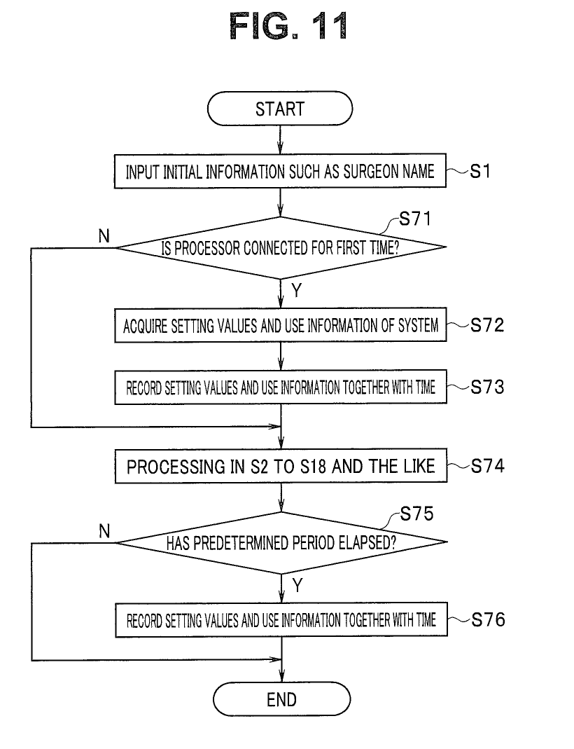

FIG. 11 briefly shows such processing.

After first step S1, in step S71, the arithmetic processing section 53 determines from a serial number of the endoscope processor 13I connected to the server 2 whether the endoscope processor 13I is connected to the server 2 for the first time.

When the endoscope processor 13I is connected to the server 2 for the first time, in step S72, the arithmetic processing section 53 acquires the setting values and the use information of the endoscope system 61 including the endoscope processor 13I from the CPU 43 of the endoscope processor 13I.

In next step S73, the arithmetic processing section 53 records the acquired setting values and the acquired use information in the recording section 51 together with time when the setting values and the use information are acquired (or the endoscope processor 13I is connected) (information concerning the acquisition or the connection).

After step S73, the arithmetic processing section 53 performs the processing in steps S2 to S18, the processing in S2 to S4 to S30 and S31, or the processing in S2 to S4 to S32 and S33 in FIG. 8. In FIG. 11, processing following step S73 is briefly shown in step S74. Note that, in step S71, in the case of a determination result that the endoscope processor 13I is not connected to the server 2 for the first time, the arithmetic processing section 53 shifts to processing in step S74.

In step S75 following step S74, the arithmetic processing section 53 determines whether a predetermined period has elapsed from the latest time when information is recorded in the recording section 51 in the endoscope system 61 connected to the server 2. Note that the predetermined period may be set to, for example, a period of approximately one week or may be set to a period of approximately a shorter number of days.

In the case of a determination result that the predetermined period has elapsed, in next step S76, the arithmetic processing section 53 records the setting values and the use information of the endoscope system 61 including the endoscope processor 13I in the recording section 51 together with time when the setting values and the use information are acquired (information concerning the acquisition). The recording section 51 saves the information recorded in the recording section 51 for a predetermined time (e.g., approximately one year or several years) set by the surgeon or set in advance.

In other words, the server 2 periodically acquires, from the respective endoscope processors 13I connected to the server 2, setting values and use information of the endoscope system 61 including setting values of the respective endoscope processors 13I and records the setting values and the use information in the recording section 51.

On the other hand, in the case of a determination result that the predetermined period has not elapsed in step S76, the arithmetic processing section 53 ends the processing shown in FIG. 11 without performing the processing in step S76.

According to this embodiment in which the operation is performed as explained above, even when a failure occurs or characteristics of a model are changed, it is possible to easily set a new endoscope processor to setting content corresponding to an old endoscope processor on the basis of use information of the old endoscope processor.

According to this embodiment, the recording section 51 is configured to record the history information. Therefore, when the surgeon sets a new endoscope processor, it is possible to select setting to setting values close to latest setting values corresponding to an examination region or organ on which an examination (or an observation) is about to be performed. Because the history information attached with the use time is recorded, it is also possible to select setting to setting values other than the latest setting values.

According to this embodiment, the arithmetic processing section 53 calculates recommendation values and records the calculated recommendation values in the recording section 51. Therefore, when the surgeon sets a new endoscope processor, it is also possible to select setting to setting values close to recommendation values corresponding to an examination region or organ on which an examination (or an observation) is about to be performed.

According to this embodiment, it is also possible to select setting in detail in a variably settable parameter range in the endoscope processor and briefly setting as in a menu range in which a variable range of parameters is narrow.

Note that, in the specification explained above, it is evident that, for example, setting values in an endoscope system include, for example, setting values in an endoscope processor configuring the endoscope system. The same applies to words other than the setting values.

Note that modifications obtained by, for example, omitting a part of the functions and the processing in the embodiment explained above and changing the order of the processing also belong to the present invention.

What is claimed is:

1. A setting changing apparatus for endoscope comprising:
a connecting section to which a first endoscope processor and a second endoscope processor are connected, the first endoscope processor being configured to perform first image processing on an image obtained by observing a first examination region in a subject to generate a first image and perform second image processing, in which a parameter different from a parameter in the first image processing is used, on an image obtained by observing a second examination region, which is an organ different from the first examination region, inside the subject to generate a second image and the second endoscope processor being configured to perform image processing on an inputted image to generate a third image;
a recording section configured to record, among information inputted from the first endoscope processor connected to the connecting section, the first image and information concerning the first examination region in association with each other and further record the second image and information concerning the second examination region in association with each other; and
an arithmetic processing circuit configured to change a parameter of the image processing in the second endoscope processor connected to the connecting section, wherein when examination of the first examination region is designated in the second endoscope processor, the arithmetic processing circuit compares the first image and the third image recorded in the recording section and changes the parameter of the image processing in the second endoscope processor to be close to a parameter of the first image processing and, when examination of the second examination region is designated in the second endoscope processor, the arithmetic processing circuit compares the second image and the third image recorded in the recording section and changes the parameter of the image processing in the second endoscope processor to be close to a parameter of the second image processing.

2. The setting changing apparatus for endoscope according to claim 1, wherein, when the examination of the first examination region is designated in the second endoscope processor, the arithmetic processing circuit compares the first image and the third image and changes, on the basis of a result of the comparison, the parameter of the image processing in the second endoscope processor such that image information configuring the third image is equal or substantially equal to image information configuring the first image and, when the examination of the second examination region is designated in the second endoscope processor, the arithmetic processing circuit compares the second image and the third image and changes, on the basis of a result of the comparison, the parameter of the image processing in the second endoscope processor such that the image information configuring the third image is equal or substantially equal to image information configuring the second image.

3. The setting changing apparatus for endoscope according to claim 2, wherein the first image processing performed on the image obtained by observing the first examination region inside the subject is performed using a first setting value, the second image processing performed on the image obtained by observing the second examination region inside the subject is performed using a second setting value, and the parameter of the image processing in the second endoscope processor is a third setting value.

4. The setting changing apparatus for endoscope according to claim 3, wherein the first image has a first feature value, the second image has a second feature value, the third image has a third feature value, and the arithmetic processing circuit determines whether a deviation amount between the first feature value and the third feature value or a deviation amount between the second feature value and the third feature value is equal to or smaller than a predetermined threshold and changes a parameter of the image processing changeable in the second endoscope processor such that the deviation amount is equal to or smaller than the predetermined threshold.

5. The setting changing apparatus for endoscope according to claim 1, wherein the recording section records, at every predetermined period, an image generated when a predetermined examination region in the subject is observed and information concerning the predetermined examination region in association with each other.

6. The setting changing apparatus for endoscope according to claim 4, wherein the recording section records, in advance, a first reference image serving as the first image generated in a first parameter for generating the first setting value respectively set in a plurality of first circuits having functions different from one another configuring the first endoscope processor and a second reference image serving as the third image generated in a second parameter at a time when the parameter for generating the third setting value respectively set in a plurality of second circuits having functions different from one another configuring the second endoscope processor is a predetermined parameter value.

7. The setting changing apparatus for endoscope according to claim 6, wherein the arithmetic processing circuit sets a first histogram representing a number of appearances distribution of a component image forming the first reference image with respect to a signal value and a second histogram representing a number of appearances distribution of a component image forming the second reference image with respect to a signal value respectively as the first feature value and the third feature value and determines whether an absolute value of an integrated value of a difference between the first histogram and the second histogram is a deviation amount equal to or smaller than the predetermined threshold in at least a plurality of representative signal values.

8. The setting changing apparatus for endoscope according to claim 3, further comprising a server including the recording section and the arithmetic processing circuit, wherein the server periodically acquires, from the first endoscope processor or the second endoscope processor connected to the server via the connecting section, a setting value and use information including information concerning examination region information in the first endoscope processor or a setting value and use information in the second endoscope processor and performs control to record the setting value and the use information in the recording section.

9. The setting changing apparatus for endoscope according to claim 8, wherein the server returns, in response to a transmission request for the first setting value or the third setting value recorded in the recording section from the first endoscope processor or the second endoscope processor connected to the server via the connecting section, information concerning the first setting value or the third setting value, transmission of which is requested, to the first endoscope processor or the second endoscope processor side.

10. The setting changing apparatus for endoscope according to claim 8, wherein, when information concerning a currently set setting value is transmitted from the first endoscope processor or the second endoscope processor connected to the server via the connecting section, the server performs control to record the transmitted information concerning the setting value in the recording section.

11. The setting changing apparatus for endoscope according to claim 8, wherein the server periodically acquires, from an endoscope processor, which is either the first endoscope processor or the second endoscope processor, connected to the server via the connecting section, information concerning a setting value of the endoscope processor, an endoscope used with the endoscope processor, models of the endoscope processor and the endoscope, a serial number of the endoscope processor, a name of a facility in which the endoscope processor is set, a name of a user who uses the endoscope processor, and information concerning a number of times of use forming the use information of the endoscope processor, records the information in the recording section with time information acquired as recording information, and further performs control to transmit the recording information, transmission of which is requested from the endoscope processor side, to the endoscope processor side.

12. The setting changing apparatus for endoscope according to claim 4, wherein the arithmetic processing circuit includes a selecting section configured to select one of processing for changing a value of the parameter within a parameter range changeable in the second endoscope processor and generating the third setting value, the deviation amount of which is equal to or smaller than the predetermined threshold, and processing for changing a value of a parameter within a menu range narrower than the parameter range and generating the third setting value, the deviation amount of which is equal to or smaller than the predetermined threshold.

13. The setting changing apparatus for endoscope according to claim 4, wherein, as the first setting value, the arithmetic processing circuit sets a setting value satisfying, when an organ at a time when an endoscopic examination using the second endoscope processor is performed is designated as first use information, a condition under which the designated organ is examined and further sets a setting value at a time when the endoscopic examination using the second endoscope processor is performed in a period designated as second use information and generates the third setting value.
